(19) United States
(12) Patent Application Publication
(10) Pub. No.: US 2026/0098802 A1
(43) Pub. Date: Apr. 9, 2026

(54) SYSTEMS AND METHODS FOR AQUEOUS NITRATE DETECTION

(71) Applicant: University of Miami, Miami, FL (US)

(72) Inventors: Daniel Furuta, Minneapolis, MN (US); Jiayu Li, Miami, FL (US)

(21) Appl. No.: 19/354,062

(22) Filed: Oct. 9, 2025

Related U.S. Application Data
(60) Provisional application No. 63/705,221, filed on Oct. 9, 2024.

(51) Int. Cl. G01N 21/31; G01N 1/14; G01N 21/33; G01N 21/94; G01N 33/18

(52) U.S. Cl. CPC G01N 21/314; G01N 1/14; G01N 21/33; G01N 21/94; G01N 33/182; G01N 2021/3181

(57) ABSTRACT

Nitrate is a major water pollutant with significant environmental and human health effects. Current field-deployable nitrate sensors are expensive, limiting monitoring and management efforts. The instant disclosure provides low-cost, field-deployable apparatuses, systems, and methods for analyzing water samples to improve pollution and analyte monitoring leading to enhanced watershed management.

### RELATED APPLICATIONS

This application claims priority and the benefit of U.S. provisional application No. 63/705,221 filed Oct. 9, 2024, the entire content of which is hereby incorporated by reference.

### FIELD

This application is directed to low-cost field deployable sensors for detecting one or more chemical species in an aqueous environment, more particularly to field deployable sensors for detecting nitrate.

### BACKGROUND

In-situ nitrate sensing is important for both research and regulatory monitoring, particularly in the fields of agriculture, wastewater treatment, and other similar sources. However, currently available nitrate sensors are expensive, which limits the spatiotemporal resolution of monitoring efforts.

Excess nitrate from agricultural fertilizer runoff, among other major sources, is a significant pollutant and threat to human health and the ecological system, including but not limited to local watersheds, freshwater bodies, and coastal regions. This excess nutrient is a contributor to eutrophication and harmful algal blooms in freshwater systems, as well as a primary driver of hypoxic dead zones for aquatic lives in coastal waters. Nitrate pollution not only threatens freshwater, coastal, and marine ecosystems but also poses risks to drinking water safety and human health, biodiversity, and economic activities, including fisheries and tourism.

The extensive use of synthetic fertilizers since the mid-1900s has led to significant increases in nitrate levels in streams and rivers, exacerbating water quality issues. In the Mississippi/Atchafalaya River Basin (MARB), agricultural nonpoint source pollution is responsible for approximately 70% of the nitrate and phosphorus loads transported to the Gulf of Mexico, contributing to the formation of one of the world's largest hypoxic zones.

Despite substantial efforts to reduce nitrate pollution, including the establishment of state-specific Total Maximum Daily Loads (TMDLs) for impaired waterbodies and the formation of the federal Hypoxia Task Force (HTF) in 1997, the goal of reducing the five-year average hypoxic zone in the Gulf region to less than 5,000 square kilometers remains unmet. Billions of dollars have been invested, but the issue persists, underscoring the urgent need for more effective strategies to control nitrate loss from agricultural watersheds and improve water quality.

The lack of sufficient measurement and monitoring data, particularly in terms of temporal and spatial coverage, is a major challenge for nitrate management. Accurate, high spatiotemporal-resolution nitrate monitoring is crucial for management due to the complex dynamics of nitrate in ecosystems. Nitrate sources can vary significantly across a region and throughout different seasons due to changes in fertilizer application, crop growth stages, soil types, weather and precipitation, and other parameters. As a result, the frequency and intensity of these sources are difficult to predict, posing challenges for nitrate models.

The movement of water through a watershed can connect diverse ecosystems, including agricultural fields, urban areas, forests, and wetlands. Nitrate levels in one part of the watershed can travel through the entire system, including groundwater and surface water bodies, driven by precipitation, snowmelt, irrigation, and surface runoff, with complex interactions along the way.

Nitrate measurements in watersheds provide the foundation for understanding and managing nutrient loss. Given nitrate distribution's complexity and highly dynamic nature, robust spatiotemporal coverage of measurements is critical. If temporal data is insufficient, short but significant nitrate peaks may be missed, hindering timely adjustments to management practices. Similarly, inadequate spatial coverage can fail to capture the variability across the watershed, limiting the effectiveness of agricultural management strategies.

The difficulty of acquiring high-resolution data is primarily due to the expense of current sensing technologies. Conventional methods involve collecting water samples in the field and sending them to a lab for analysis. While this approach does not require any expensive field equipment, it does require significant labor and infrastructure for sample collection and analysis, which is prohibitive if attempting to capture a high spatiotemporal resolution.

Commercially available in-situ nitrate sensors have gained popularity as an alternative to traditional sampling techniques. These sensors offer the advantage of providing long-term continuous measurements with higher temporal resolution and reduced labor costs compared to sampling methods. However, they remain costly, with complete systems priced in the tens of thousands of dollars. The high capital expense of these devices makes deploying a large number infeasible, which in-turn, reduces the spatiotemporal resolution of monitoring efforts.

Academics, institutions, and various organizations are making ongoing efforts to map the spatiotemporal distribution of nitrate concentrations using existing sensing techniques. The Environmental Working Group (EWG) has compiled historical data on nitrate monitoring in the Upper Mississippi River Basin. The U.S. Geological Survey (USGS) WaterQualityWatch program provides continuous real-time surface water nitrate concentration data, updated hourly. Additionally, the NSF-supported NEON Aquatic Sampling Strategy collects data from 34 aquatic sites across the U.S., measuring nitrate concentrations using the SUNA sensor (OTT Hydromet), with more data expected to become publicly available in the coming years.

However, current nitrate monitoring methods are limited in their ability to capture the complex, dynamic nature of nitrate pollution leading to inadequate spatiotemporal coverage. The general inventive concepts seek to address this issue with the systems and methods described herein.

### SUMMARY

The present disclosure provides devices, systems, and methods for optical fluid analysis. Embodiments include a field-deployable sampling and analysis device. A fluid may be pumped/routed through the sampling system and data, including real-time data, is collected from the fluid via the analysis portion of the device.

The general inventive concepts are based, in part, on the recognition that a modular system for analyte detection and measurement can be developed at a lower cost and with reduced maintenance needs relative to expensive and/or unwieldy conventional systems. Such a system can provide better timescale resolution to detect harmful analytes as they change, allowing for improved responses thereto.

In one sense, the general inventive concepts seek to improve current measurement and monitoring of analytes in water sources both in terms of responsiveness (i.e., up to the minute measuring and communication) and in terms of geographical coverage (i.e., a more specific and granular map of the area under observation). This and other needs are addressed by the devices, systems, and methods described herein.

Provided below is a disclosure for a low-cost, modular, analyte sensing device/system. The low-cost affordability enables large-scale deployment of an array of devices, which improves spatial coverage and allows the user to capture variability across different watershed types and scales. Additionally, the sensor has short sampling intervals to allow for accurate tracking of dynamic changes in nitrate concentrations over time. Further, because the device is relatively low maintenance, the need for frequent on-site visits and monitoring is reduced, further lowering the overall cost.

In certain embodiments, the general inventive concepts describe a field-deployable device for analyzing one or more analytes in an aqueous source. The device comprises a unit that sits on dry land, with a pump configured to pull sample water (e.g., surface or ground water) into a sample chamber. The unit additionally comprises a first LED disposed opposite a first photodiode, such that a first path is formed. A first focusing lens is disposed of centrally between them. A second LED is disposed of opposite a second photodiode, forming a second path and is oriented such that the second path is perpendicular to the first path. A second focusing lens is disposed centrally between the second LED and second photodiode. The sample chamber is disposed at the intersection of the first path and second path, such that the lights emitted by the first LED and second LED pass through the focusing lenses and hit the sample chamber before continuing to their respective photodiode. Both LEDs and both photodiodes are each in communication with a circuit board. The first LED is configured to emit a light at a baseline reference for the absorbance of the water not including a specific analyte (for example, due to turbidity, colored organic matter, or other possible particulates). The second LED is configured to emit a light on a spectrum sensitive to a specific analyte, to the extent possible given the technical limitations of reasonably priced LEDs. Nitrate, for example, absorbs UV light from 200 nm to about 235 nm, but not substantially at above 250 nm, e.g., 250 nm to about 275 nm. In this way, by measuring how much light is absorbed by each photodiode a user can determine how much analyte is present in the sample water by correcting based on the light detected from each emitter. In other words, one aspect of the general inventive concepts is measuring differential absorption between the two measured wavelengths.

In certain embodiments, the general inventive concepts describe a field-deployable device for analyzing one or more analytes in an aqueous source, the device comprising a unit that sits on dry land, comprising a sample chamber and a pump configured to pull sample water into the sample chamber; a first LED disposed opposite a first photodiode, such that a first path is formed therebetween; a first focusing lens disposed between the first LED and the first photodiode; a second LED disposed opposite a second photodiode, forming a second path and oriented such that the second path is perpendicular to the first path; a second focusing lens is disposed between the second LED and second photodiode; the sample chamber is disposed at an intersection of the first path and second path, such that light emitted by the first LED and light emitted by the second LED pass through the focusing lenses and the sample chamber before continuing to the first photodiode and second photodiode; both LEDs and both photodiodes are each in communication with a circuit board; the first LED is configured to emit light at a predetermined wavelength sensitive to a specific analyte the second LED is configured to emit light at a predetermined baseline wavelength for the absorbance of the water not including a specific analyte.

In certain embodiments, the general inventive concepts describe a field-deployable array for monitoring analytes in a water source, where the field-deployable array comprises at least two spatially separated field-deployable devices for monitoring analytes in a water source as described herein.

In certain embodiments, the general inventive concepts describe a method for real-time detection of at least one analyte in a water source, the method comprising providing a system for measuring at least one water-born analyte comprising a dry land unit, a pump, and a sample chamber, pumping water from a water source into the sample chamber, positioning the sample chamber at an intersection of two UV sensors, reading the sample based on differential UV optical absorption from the two UV sensors, and emptying the sample chamber once the reading has been taken.

In certain embodiments, the general inventive concepts describe a method for analyzing a confined volume of water within a prescribed region of a sample chamber with an optical nitrate sensor, comprising pumping a ground water sample from a water source; confining the ground water sample in a sample chamber positioned within a unit that sits on dry land, wherein the sample chamber sits at an intersection between a first light path from a first light emitter and a second light path from a second light emitter, wherein the second light path is oriented perpendicular to the first light path; generating a first LED signal at about 220 nm to about 235 nm with a first LED, and a second LED signal in a range of about 250 nm to about 275 nm with a second LED, wherein the first LED signal and the second LED signal traverse the confined volume of water within the prescribed region of the sample chamber; sensing with a measuring photodiode the first LED signal and the second LED signal; providing photodiode measured signal containing information about a nitrate absorption of the water related to the first LED signal and the second LED signal; receiving with a signal processing module the photodiode measured signal containing information from the first LED and the second LED; calculating the concentration of nitrates dissolved in the water by compensating the concentration of the absorption at about 220 nm to about 235 nm for the absorption at about 250 nm to about 275 nm in order to determine a level of nitrates in the water.

### BRIEF DESCRIPTION OF THE DRAWINGS

The general inventive concepts, as well as embodiments and advantages thereof, are described below in greater detail, by way of example, with reference to the drawings in which:

FIG. 1A shows an assembled sensor according to the general inventive concepts.

FIG. 1B is a schematic of an exemplary optical path in a sensor according to the general inventive concepts.

FIG. 1C is an image showing the sensor interior in the same orientation as the schematic.

FIG. 2 is an image showing a prototype of the inventive sensor system deployed in a test experiment.

FIG. 3A is a graph showing LED voltage versus temperature.

FIG. 3B is a graph showing optical path response versus temperature.

FIG. 4A is a graph showing sensor outputs during pre-experiment calibration.

FIG. 4B is a graph showing sensor outputs during post-experiment calibration.

FIG. 5 is a graph showing the pre-experiment and post-experiment clean water calibrations on averaged data, fit with third-order polynomials. Each point represents all data for that nitrate level across all recorded temperature levels.

FIG. 6A is a graph showing exemplary sensor performance on pre-experiment clean water calibration without averaging.

FIG. 6B is a graph showing exemplary sensor performance on pre-experiment clean water calibration with averaging.

FIG. 7 is a graph showing the exemplary sensor response as defined previously, temperature, and nitrate levels as measured by the reference sensor.

FIG. 8A is a graph showing exemplary sensor performance in dirty water, using the calibration equation developed in clean water.

FIG. 8B is a graph showing exemplary sensor performance in dirty water, using the calibration equation developed in dirty water experiments.

FIG. 9A is a graph showing sensor performance without averaging.

FIG. 9B is a graph showing sensor performance with averaging over 10 minutes.

FIG. 9C is a graph showing sensor performance with averaging over 30 minutes.

FIG. 9D is a graph showing sensor performance with averaging over 120 minutes.

### DESCRIPTION

Several illustrative embodiments will be described in detail with the understanding that the present disclosure merely exemplifies the general inventive concepts. Embodiments encompassing the general inventive concepts may take various forms and the general inventive concepts are not intended to be limited to the specific embodiments described herein.

While various exemplary embodiments are described or suggested herein, other exemplary embodiments utilizing a variety of methods and materials similar or equivalent to those described or suggested herein are encompassed by the general inventive concepts.

Unless defined otherwise, all technical and scientific terms used herein have the same meaning as commonly understood by one of ordinary skill in the art to which the invention belongs.

Nitrate is a major water pollutant with significant environmental and human health effects. Current field-deployable nitrate sensors are expensive, limiting monitoring and management efforts. However, timely, high resolution sensor data that tracks analytes like nitrate is rarely available at a local small watershed scale, and sensor deployment is prohibitively expensive at a field scale. This lack of granularity and specificity makes it challenging for smaller stakeholders to use sensor data to make responsive adjustments to agricultural and water management practices.

Thus, there exists a need for low-cost sensors/arrays that may provide reliable field data with enhanced spatial and temporal coverage. This type of sensor would have multiple benefits across multiple areas. For example, it would help deepen the understanding of nitrate sources, transformation, transport, and leaching within ecosystems, as well as the associated health and environmental impacts. Additionally, this type of sensor would also provide stakeholders a method of acquiring the necessary information to make decisions on fertilizer use and land management, ultimately reducing nitrate losses and improving watershed health. Furthermore, this type of sensor would give water resource managers and environmental agencies more effective tools for tracking nitrate pollution and improving water quality, which in turn would support efforts to reduce excess nitrate, advance precision agriculture, and promote sustainability. This type of sensor would also allow for the aggregation of data, which could allow policymakers to utilize the data for more informed regulation and mitigation strategies. Overall, sensors/arrays according to the general inventive concepts would allow researchers to gain valuable insights into nitrate dynamics, while also allowing rural communities dependent on natural waters (e.g., surface and groundwater) to improve the quality of their drinking water, thereby reducing health risks such as methemoglobinemia.

In certain exemplary embodiments, the system is embodied by a two-wavelength optical spectrometer measuring light absorption at nominal wavelengths of about 220 nm to about 235 nm and about 250 nm to about 275 nm. Nitrate absorbs around 200 to 235 nm light but has little absorption above 250, e.g., at 275 nm. The general inventive concepts use the ratio between absorption at the two wavelengths to compensate—for/reduce the effect of turbidity and potentially interfering analytes (e.g., dissolved organic content, DOC). In other words, one aspect of the general inventive concepts is measuring differential absorption between the two measured wavelengths (i.e., differential absorption between the first absorption at about 220 nm to about 235 nm and the second absorption at about 250 nm to about 275 nm), see e.g., equations 1-5. The devices and systems of the general inventive concepts differ from conventional systems in the use of separate optical paths for the two measured wavelengths, which reduced component costs. Commonly, commercially available sensors use broadband UV emitters, with beamsplitters and filters to select the different wavelengths of interest. Thus, an improvement that the general inventive concepts deliver is a work-around that eliminates the need for expensive beamsplitters and filters to select the necessary wavelengths. The device according to the general inventive concepts avoids the need for optical components beyond one focusing lens per path, though the general inventive concepts do utilize a photodiode for each optical path. As the cost of an additional photodiode is low compared with the cost of additional lenses or other optics, Applicants decided the tradeoff was acceptable due to the coast savings while not adding substantial complexity to the device.

In certain embodiments, the general inventive concepts describe a field-deployable device for analyzing one or more analytes in an aqueous source. The device comprises a unit that sits on dry land, with a pump configured to pull sample water (e.g., pump surface or ground water) into a sample chamber. The unit additionally comprises a first LED disposed opposite a first photodiode, such that a first path is formed. A first focusing lens is disposed of centrally between them. A second LED is disposed of opposite a second photodiode, forming a second path and is oriented such that the second path is perpendicular to the first path. A second focusing lens is disposed centrally between the second LED and second photodiode. The sample chamber is disposed at the intersection of the first path and second path, such that the lights emitted by the first LED and second LED pass through the focusing lenses and hit the sample chamber before continuing to their respective photodiode. Both LEDs and both photodiodes are each in communication with a circuit board. The first LED is configured to emit a light at a baseline reference for the absorbance of the water not including a specific analyte (for example, due to turbidity, colored organic matter, or other possible particulates). The second LED is configured to emit a light on a spectrum sensitive to a specific analyte, to the extent possible given the technical limitations of reasonably priced LEDs. Nitrate, for example, absorbs UV light from 200 nm to about 235 nm, but not substantially at above 250 nm, e.g., 250 nm to about 275 nm. In this way, by measuring how much light is absorbed by each photodiode a user can determine how much analyte is present in the sample water by correcting based on the light detected from each emitter. In other words, one aspect of the general inventive concepts is measuring differential absorption between the two measured wavelengths.

In certain embodiments, the general inventive concepts describe a field-deployable device for analyzing one or more analytes in an aqueous source, the device comprising a unit that sits on dry land, comprising a sample chamber and a pump configured to pull sample water into the sample chamber; a first LED disposed opposite a first photodiode, such that a first path is formed therebetween; a first focusing lens disposed between the first LED and the first photodiode; a second LED disposed opposite a second photodiode, forming a second path and oriented such that the second path is perpendicular to the first path; a second focusing lens is disposed between the second LED and second photodiode; the sample chamber is disposed at an intersection of the first path and second path, such that light emitted by the first LED and light emitted by the second LED pass through the focusing lenses and the sample chamber before continuing to the first photodiode and second photodiode; both LEDs and both photodiodes are each in communication with a circuit board; the first LED is configured to emit light at a predetermined wavelength sensitive to a specific analyte the second LED is configured to emit light at a predetermined baseline wavelength for the absorbance of the water not including a specific analyte.

The instant devices and systems differ from conventional measurement and analysis systems in that they are positioned on dry land near the water being sampled. This arrangement utilizes a pump to draw water from the water source and deliver it to the sample/measurement chamber. This has the advantage of not requiring conventional and expensive configuration or components such as waterproofing, gaskets, optical windows, and similar components required for submerged deployment. We demonstrate the sensor in a laboratory setting in this experiment but moving it to the field only requires adding a battery, solar charger, and weatherproof enclosure.

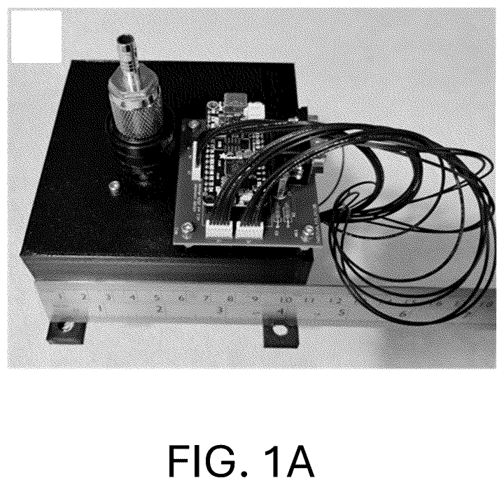

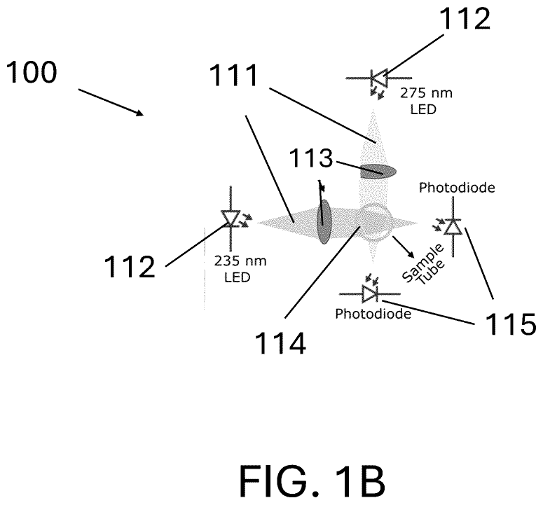

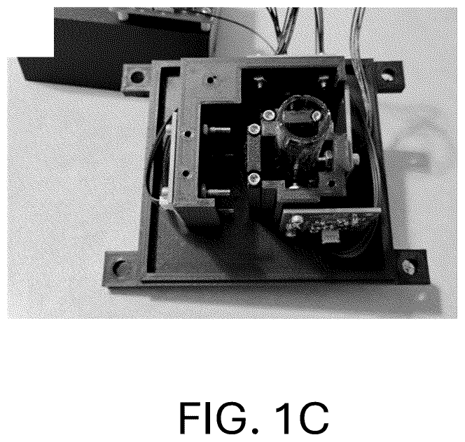

As can be seen from FIG. 1B, the sensor 100 of the inventive device is built around two optical paths 111 substantially perpendicular to one another, which are identical except for LED wavelength. In each path, an LED 112 emits light which is focused by a biconvex lens 113 through a tube 114 containing the water sample (sample container) onto a photodiode detector 115. While other optical paths and emitter arrangements are possible and fall within the scope of the general inventive concepts, Applicants selected the orthogonal relationship to avoid potential optical interference between the emitters and their respective photodiodes. Also, the instant device arrangement provides benefits for measuring differential absorption between the two measured wavelengths.

In the embodiments shown in the Examples, Applicants calculated the placement of the optical elements using OSLO (Lambda Research Corp., Westford MA, USA).

In the exemplary embodiments shown in the Examples, the sample chamber takes the form of a cylinder thereby the system has different focal points in different planes. In the exemplary embodiments Applicants place the photodiode at the closest focal point, and so the image of the LED at the detector is a line and not a point. This astigmatism could easily be corrected with the addition of cylindrical lenses, but Applicants elected to accept the aberration due to cost increases, though the use of cylindrical lenses to compensate for the cylindrical sample chamber is within the scope of the general inventive concepts.

In the exemplary embodiments shown in the Examples, the lenses are fused silica (LB4854, Thorlabs Inc., Newton NJ, USA), and the sample tubing is 16 mm OD, 13 mm ID quartz glass (Technical Glass Products Inc., Painesville OH, USA). We chose a photodiode with response from 220 nm to 280 nm (008-2171-112, Advanced Photonix, Camarillo CA, USA) to avoid interference from fluorescence or visible light leakage. We selected 235 nm and 275 nm LED emitters (MT2350D, Marktech Optoelectronics, Latham NY, USA and EOLD-275, EPIGAP OSA photonics, Berlin, Germany respectively). The lenses and LED emitters constitute the majority of the cost of the sensor. Importantly, photodiodes should be selected to avoid picking up interference from fluorescing due to UV light and/or from, for example, daylight if the device is not fully sealed.

In the exemplary embodiments shown in the Examples, Applicants designed the optomechanical assembly in FrecCAD and printed it in PETG filament using a 3D printer (Prusa Research, Prague, Czechia). We used inexpensive fittings designed for consumer computer water cooling (HardTube, Alphacool International GmbH, Braunschweig, Germany) to connect the plumbing.

The disclosed optical design is low cost and simple but requires additional calibration relative to more expensive conventional approaches. The beam through the sample tubing is not collimated and does not have a single path length, and so we violate the assumptions of the Beer-Lambert law and require a different calibration approach. Our printed sensor assembly is considerably less expensive than machined metal optomechanics, but it also has lower rigidity and durability. The uncorrected astigmatism of our design due to the cylindrical sample container also reduces the amount of energy focused on the detector.

Each UV emitter (e.g., LED) and photodiode in the sensor mounts on a separate printed circuit board (PCB), all of which connect via cables to a controller PCB. The controller PCB then connects to a main board which acts as a power supply and provides telemetry. The sensor has two LED boards, two photodiode boards, one controller board, and one main board, all of which were designed for the instant application.

The LED boards provide drivers to switch the LEDs on and off. We set the 235 nm LED drive to 40 mA and the 275 nm LED to 20 mA of current; both values were chosen conservatively within the diodes' specifications to avoid unnecessary heating. Each LED board digitizes the LED voltage and current using a 16-bit analog to digital converter, and each also contains a temperature and relative humidity sensor to monitor operating conditions.

The photodiode board amplifies and digitizes the small output current from the photodiode. The photodiode connects to a transimpedance amplifier, designed with a DC gain of 2 V per 100 nA, which is then digitized by a 16-bit analog to digital converter. We chose the amplifier gain to give a response of around 1 V when sensing distilled water without added nitrate. The small output signal from the photodiode required careful circuit board layout.

The devices showed good performance on averaged data in the clean water calibration testing, with RMSE-0.67 mg/L of nitrate. This result is comparable with some commercially available units. As expected, the device had diminished performance in dirty water with RMSE=2.75 mg/L, but this value improved to 1.46 mg/L with averaging. This result could be improved with further development but is already sufficient to capture trends in agricultural runoff or similar sources where absolute accuracy is often less important than trends and overall durability and case of deployment and maintenance of the system.

Initial measurements indicated a temperature effect on the device's optoelectronics due to the differing responses of LEDs and photodiodes at different junction temperatures. However, this effect was corrected by measuring operating temperatures and then applying the correction curve described below. After this correction we did not see a significant temperature effect on the sensor's performance.

The devices according to the general inventive concepts, performed better with averaging than without in both our clean and dirty water experiments, and the error did not appear to be strongly correlated with any particular parameter. Our reading protocol, in which the sensor took ten values per optical path and retained the maximums, was based on prior experience but was likely insufficient. As the sensor's measurement error appeared to largely be due to random noise, modifying the reading protocol to sample a larger number of values per reading would likely improve the sensor's accuracy without requiring hardware design changes. In certain exemplary embodiments, the general inventive concepts measure the absorbance at least 10 times per sample, including at least 50 times per sample, including 10 to 1000, including 10 to 900, including 10 to 800, including 10 to 650, including 10 to 500, including 10 to 375, including 10 to 150, including 50 to 100, including 100 to 100, including 250 to 1000, including 425 to 100, including 625 to 100, and including about 1000 times per sampling (i.e., per instance of the pump pumping the water from the water source and delivering the sample water to the sample chamber).

The inventive device/sensor clearly captured nitrate trends in our dirty water test, but with noise and some bias. The calibration from clean water did not transfer to nitrate levels in the dirty water experiment, suggesting that matrix effects play an important role in the sensor response. We will examine the sensor's behavior with controlled levels of turbidity, dissolved organic carbon (DOC), and non-target contaminants as a future step to address this challenge; likely the device's performance can be improved with further calibration work, or alternately by adding an additional wavelength to better characterize other contaminants. Interference from DOC is a well-known challenge for optical nitrate sensors, and so addressing this sensitivity is an expected and critical next step. In certain exemplary embodiments, the device further comprises an emitter (e.g., a third LED) that emits a signal at a wavelength of about 350 nm (and a corresponding detector/photodiode). Such an emitter would aid in detecting/correcting for DOC, in combination with the first and second LEDs. In certain exemplary embodiments, the device may further comprise one or more additional visible light emitters (and corresponding detectors).

Applicants' low-cost devices show clear promise for enabling affordable, dense nitrate sensor networks. The parts cost of our one-off unit was less than a tenth of the cost of current commercial sensors; with further development and production at scale we expect to maintain a substantially lower cost than currently available options. Besides enabling improved nitrate pollution monitoring, our device could easily be adapted to in-line operation for precision agriculture, hydroponics, or other process applications.

Applicants designed and built a prototype low-cost nitrate sensor at a very low cost (e.g., for under $800 USD in parts).

In certain exemplary embodiments, the general inventive concepts are embodied by a method for analyzing a confined volume of water within a prescribed region of a sample chamber with an optical nitrate sensor, comprising pumping a ground water sample from a water source; confining the ground water sample in a sample chamber positioned within a unit that sits on dry land, wherein the sample chamber sits at an intersection between a first light path from a first light emitter and a second light path from a second light emitter, wherein the second light path is oriented perpendicular to the first light path; generating a first LED signal at about 220 nm to about 235 nm with a first LED, and a second LED signal in a range of about 250 nm to about 275 nm with a second LED, wherein the first LED signal and the second LED signal traverse the confined volume of water within the prescribed region of the sample chamber; sensing with a measuring photodiode the first LED signal and the second LED signal; providing photodiode measured signal containing information about a nitrate absorption of the water related to the first LED signal and the second LED signal; receiving with a signal processing module the photodiode measured signal containing information from the first LED and the second LED; calculating the concentration of nitrates dissolved in the water by compensating the concentration of the absorption at about 220 nm to about 235 nm for the absorption at about 250 nm to about 275 nm in order to determine a level of nitrates in the water. In certain exemplary embodiments, the method comprises determining a level of nitrate according to equations 1-5, including equation 5.

In certain exemplary embodiments, the method comprises discharging the sample water from the sample chamber and pumping a second sample into the sample chamber to determine a time scale level of nitrate in the water. This may be performed iteratively (i.e., pumping, emitting, detecting, calculating, etc.). In certain exemplary embodiments, the method comprises determining a level of nitrate at more than one location, determining a level of nitrate at more than one location. In certain exemplary embodiments, the method comprises compensating for dissolved organic content by measuring, calculating, and compensating for a third LED absorption at e.g., 350 nm.

In certain exemplary embodiments, the method comprises the method comprises the measurement by determining the level of nitrates from 10 to 100 times per pumping, including from 100 times to 1000 times per pumping. In certain exemplary embodiments, the determining is averaged to determine the level of the analyte (e.g., nitrate). In certain exemplary embodiments, measuring and determining occur at the site of the device (i.e., local measurement, calculation, averaging, and/or determining). In certain exemplary embodiments, the method further comprises transmission of data (e.g., wireless, Bluetooth, or internet transmission) for measuring and determining at a location apart from the site of the device (i.e., local measurement, calculation, averaging, and/or determining occur at a centralized or otherwise distant location from the location of the device and the subject water site).

All references to singular characteristics or limitations of the present disclosure shall include the corresponding plural characteristic or limitation, and vice versa, unless otherwise specified or clearly implied to the contrary by the context in which the reference is made.

The following examples illustrate features and/or advantages of the devices, systems, and methods according to the general inventive concepts. The examples are given solely for the purpose of illustration and are not to be construed as limitations of the general inventive concepts, as many variations thereof are possible without departing from the spirit and scope of the general inventive concepts.

### EXAMPLES

Sensor read procedure: To take a reading, the system first draws a water sample into the sample chamber (tube) with the pump. The system then settles for 30 seconds to allow any large particles to sink out of the optical path. Next, the first LED (i.e., a 235 nm LED) turns on and the module measures the photodiode response and LED voltage and current. The first LDE (235 nm LED) is turned off, and the process is repeated for the second LED (i.e., a 275 nm LED). Then, the first LDE (235 nm LED) is once again turned on, and so on. We performed ten cycles per reading and then stored the maximum photodiode response for each path from all ten iterations. We chose to use the maximum values instead of averages in the hope of reducing the effect of any particles or other contamination that might affect some readings but not others; we left optimizing this reading protocol to future work.

We also recorded the levels recorded on the inactive photodiode for each LED activation (i.e., when the first LED is active, the level is recorded for the second LED despite the second LED not emitting, and vice versa), at right angles to the active LED. For example, if the 235 nm LED was active, we also recorded the level seen by the 275 nm path's photodiode. As this measurement is similar to the light scattering approach used in nephelometers for turbidity sensing, we anticipated that it could provide some insight into the particulate content of the water sample. We recorded the temperature and humidity readings from each LED board to use in correcting for environmental factors.

Per reading, we used the maximums of the ten photodiode measurements for each path and the temperature data from each LED board for the main analysis of this paper. We used the right-angle data to examine the factors influencing error in our calibration, and we used the LED voltage and current data to confirm that the recorded board temperatures were well correlated with LED junction temperature, as we will explain.

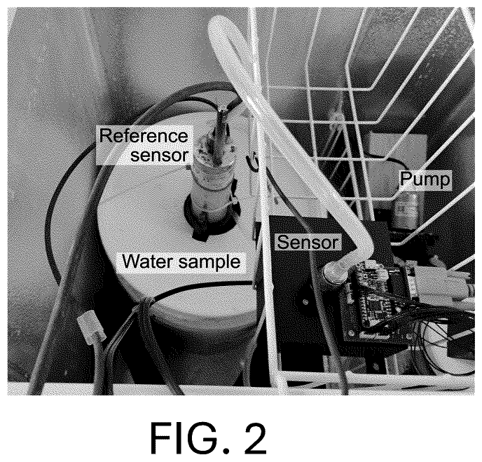

Test chamber Design: Many electronic devices show effects from changing temperatures, and optoelectronics are frequently temperature sensitive. To examine the influence of temperature on our sensor's performance, we conducted our sensor calibration using a consumer chest freezer as a temperature-controlled chamber. We placed two small heaters (seedling heat mat, Vivosun, Ontario CA, USA; unbranded aquarium heater inside a sealed jar of water) inside the chamber and used a temperature controller (ITC-308, Inkbird Tech., Shenzhen, China) to power both the heaters and the freezer to maintain a set temperature. This setup is known to maintain temperature within approximately 2° C. We placed the sensor and water samples inside the chamber for the experiments. For the dirty water experiment, described later, we also placed a reference sensor inside the sample container (ecoN, OTT HydroMet Corp., Sterling VA, USA). FIG. 2 shows the experimental setup for the dirty water experiment, with the sensor, pump, water sample, and reference sensor visible. The heaters are obscured by other components in the photo.

We found that the LED drive performed with excellent stability regardless of temperature in our temperature calibration. The variation in the current of the 235 nm LED was smaller than the resolution we could record, corresponding to less than 0.06 mA. The 275 nm LED saw a variation corresponding to approximately 0.06 mA, or around 0.15% of the drive current. We will ignore this negligible variation in LED current for the remainder of the analysis.

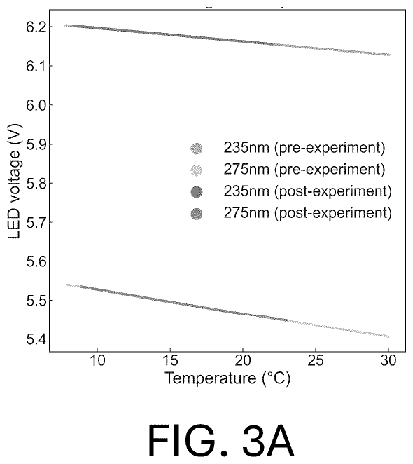

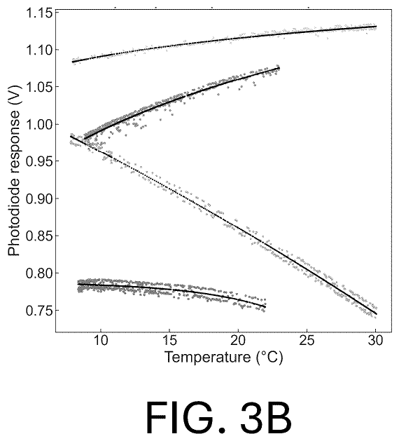

As expected from the underlying changes in semiconductor band gaps with temperature, the LED voltages and overall optical path response showed a strong connection with temperature. As seen in FIG. 3A, the LED voltage and temperature showed a nearly linear relation with each other across the temperature range we observed; linear fits for each LED voltage as functions of their respective temperature sensor readings yielded R2>0.999 and RMSE<1 mV for both LEDs. Note that FIG. 3A is a scatterplot of unprocessed, collected data, not a fit curve.

As mentioned, we had to repair the sensor prior to the dirty water experiment and performed a post-experiment recalibration on the rebuilt sensor after the experiment. The voltage and temperature relationships were essentially identical for the pre-experiment and post-experiment calibrations, as shown in FIG. 3A, indicating that the sensor repair did not affect this relationship for either LED. As the voltage and temperature readings are so closely linked, we will continue with the temperature readings and will not use LED voltages further in our analysis. We note that for future sensor revisions it appears unnecessary to include both temperature and LED voltage sensing, and either could function equivalently.

FIG. 3B shows the response of each optical path with distilled water across the temperature range for both the pre-experiment calibration and the post-experiment recalibration. We expected the LED output to decline with increasing temperature, which we observed for the 235 nm path. However, the 275 nm path showed an increase with temperature. We believe this behavior may be due to changing sensitivity of the photodiode with temperature at the long end of its response (specified to 280 nm).

The pre-experiment and post-experiment calibrations showed distinctly different curves for both optical paths. As only one photodiode was replaced, this is likely due in part to differences in optical alignment from the reassembly process. As the focal length of our optomechanical system is relatively short (the lenses we used have 20 mm focal lengths), and as we chose a printed plastic assembly to save cost over machined metal, small changes in mechanical positioning are both likely with reassembly and can have a large influence on sensor response. Increasing the rigidity of the optomechanical design will be a future priority.

If the shift in response were due to accumulated dirt in the post-experiment recalibration, we would expect the slope to remain similar and only the magnitude of the response to change; it is also unlikely that aging of the LEDs or photodiodes would result in a shift of this magnitude over the time period of the experiment. We will further explore the effects of device aging and alignment in future work.

We fit the 235 nm path's temperature response with the simple exponential decay in Eq. 1, where V is the photodiode response and T is the recorded temperature. To is a baseline temperature for each optical path, which is a fit parameter. We fit the 275 nm path logarithmically with Eq. 2. We performed both fits numerically. Our fits resulted in R2 values of 0.99 and 0.97, and RMSE values of 6.2 mV and 2.5 mV for the 235 nm and 275 nm paths respectively for the pre-experiment calibration. For the post-experiment calibration, the fits produced R2 values of 0.61 and 0.97, and RMSE values of 5.4 mV and 5.1 mV for the 235 nm and 275 nm paths respectively. The fits are shown in FIG. 3B. The fits for the pre-experiment and post-experiment calibrations both successfully captured the trends, but the different parameters suggest a need for new calibrations after system maintenance and between units.

$${V}_{235 nm} ( t ) = {a}_{235 nm} + {b}_{235 nm} {e}^{{k}_{235 nm} ( T - {T}_{0 \_ 235 nm} )} \qquad 1$$

$${V}_{275 nm} ( t ) = {a}_{275 nm} + {b}_{275 nm} log [ {k}_{275 nm} ( T - {T}_{0 \_ 275 nm} ) ] \qquad 2$$

We then implemented the simple temperature correction scheme shown in Eq. 3. First, for each photodiode, we determine a reference response in clean water at an arbitrarily chosen temperature of 10° C. using Eq. 1 or 2. Given a photodiode reading at temperature T, we then calculate the expected response for clean water at temperature T using Eq. 1 or 2. The ratio between the two is the expected shift in photodiode response due to temperature, and so we then multiply the observed reading by this ratio to yield our corrected photodiode response.

$${V}_{235 nm corrected} = {V}_{observed} \frac{{V}_{235 nm} ( 10 ° C . )}{{V}_{235 nm} ( T )} \qquad 3 a$$

$$or$$

$${V}_{275 nm corrected} = {V}_{observed} \frac{{V}_{275 nm} ( 10 ° C . )}{{V}_{275 nm} ( T )} \qquad 3 b$$

Performance in clean water: Given the collected data in distilled water with added nitrate, we first corrected the photodiode responses for temperature using Eq. 3, using the parameters derived above for each calibration. We then took the ratio between the two photodiodes' responses with Eq. 4, which we use as the sensor output S. We use the 275 nm path to establish a baseline for optical clarity of the water sample and assume that deviation from this baseline in the 235 nm path is due to absorption from nitrate. This approach ignores matrix effects from other contaminants and is intended as an initial approach to be refined in future work.

$$S = \frac{{V}_{235 nm corrected}}{{V}_{275 nm corrected}} \qquad 4$$

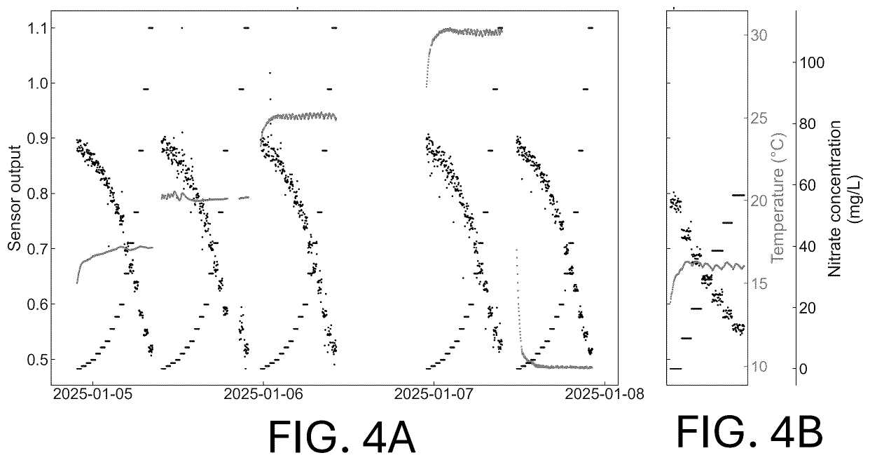

As an illustration of the clean water calibration showing the differing nitrate and temperature levels, FIG. 4 is a timeseries of the collected data. The temperature correction appears to largely remove the effect of temperature on the sensor response. The sensor shows a clear inverse relationship with nitrate levels as expected. We show both the pre-experiment calibration and the post-experiment calibration; both show a strong response to nitrate but differ in the magnitude of their response.

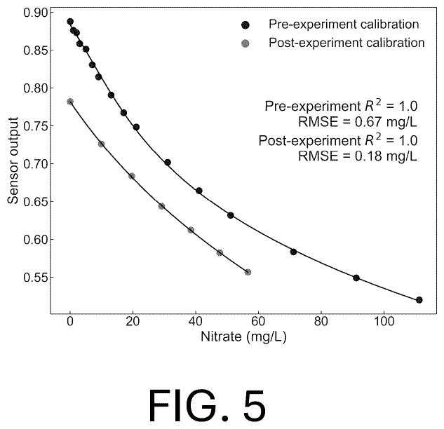

To remove the influence of noise, we then proceeded with the calibration using the data averaged across all runs within each nitrate concentration level, separately for the pre-experiment and post-experiment calibrations. We fit the data with a third-order polynomial, shown in Eq. 5, where S is the sensor output as defined in Eq. 4. As seen in FIG. 5, this produced a close fit on the pre-experiment calibration data, with RMSE of 0.67 mg/L. The fit on the post-experiment recalibration, with fewer data points, produced a similarly good fit but with different parameters, suggesting again that individual units will need to be calibrated independently, and that recalibration will be necessary after maintenance and repairs.

$$Nitrate = {aS}^{3} + {bS}^{2} + cS + d \qquad 5$$

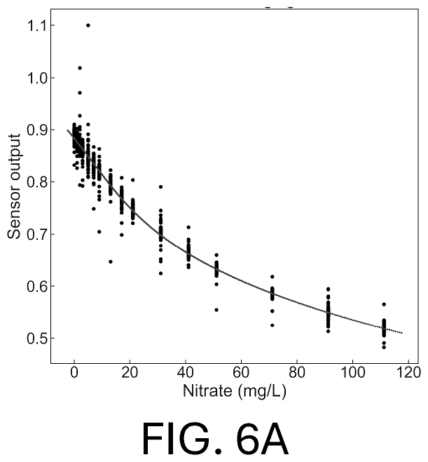

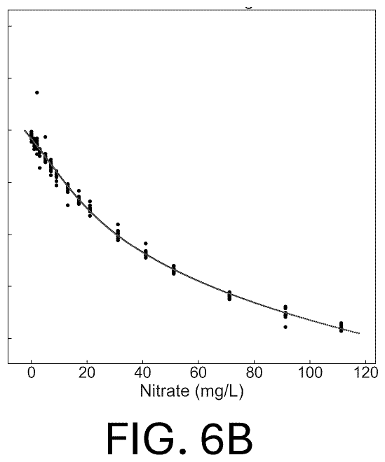

We then applied the fit to the complete dataset for the pre-experiment calibration with time averaging within each nitrate concentration level, shown in FIG. 6. The fit line is that derived from the fully averaged dataset as in FIG. 5. We examined the correlation between prediction error and the different variables and did not find scaled prediction error to be correlated with temperature, photodiode levels, or nitrate concentration with Pearson's r greater than 0.1. As the error diminishes with averaging, we believe that our results could likely be significantly improved by collecting and averaging across more readings than in the current sampling protocol even without any hardware or algorithm modifications.

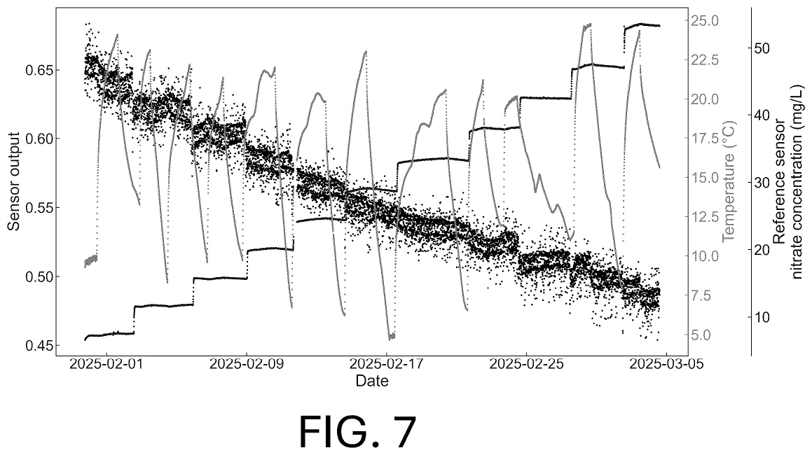

Test in dirty water: Next, we analyzed the dirty water experiment, shown as a time series in FIG. 7 with the sensor response corrected using the post-experiment calibration's temperature parameters derived in Section 3.1. The sensor readings show a clear response to nitrate levels but also contain substantial noise and variation. There is not a clear temperature response visible, suggesting that the temperature correction was successful.

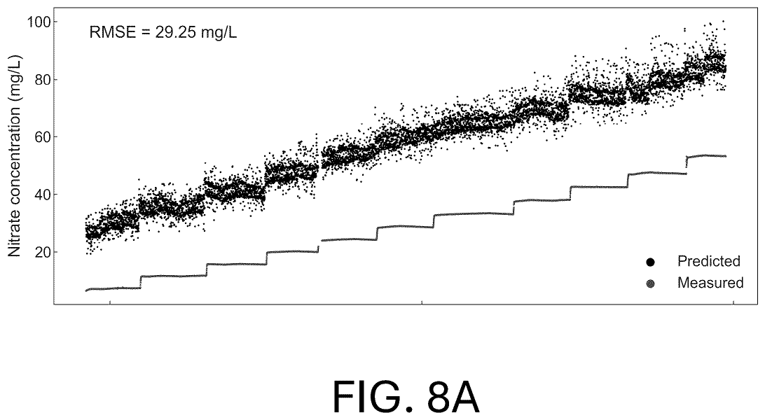

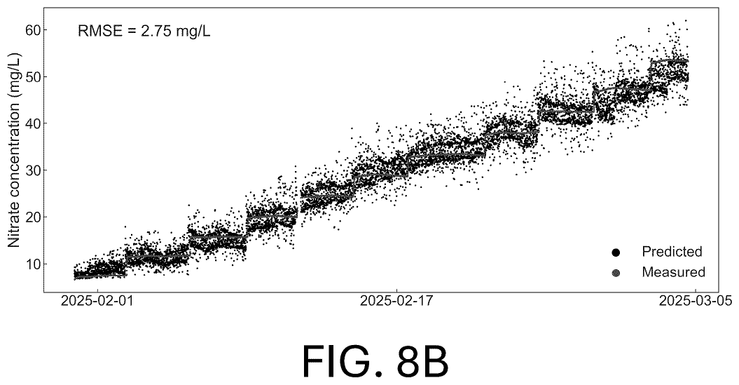

We next applied the curve from our post-experiment calibration to the dirty water dataset and found that it did not translate. As seen in FIG. 8A, the transferred calibration captures the general nitrate trend, but with incorrect levels. We then re-fit Eq. 5 on the dirty water dataset with results seen in FIG. 8B. The calibration appears to underpredict high concentrations and overpredict low concentrations, but has reasonable overall accuracy with RMSE=2.75 mg/L. This difficulty in transferring calibrations is likely due to dissolved organic carbon (DOC) present in the dirty water but not in the clean water, which has been noted as having a similar effect on some commercial sensors. This challenge could be addressed in a later revision by adding an additional wavelength to characterize DOC, or possibly by simply calibrating across a range of DOC levels.

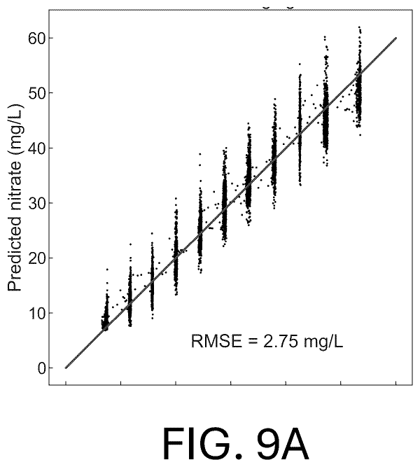

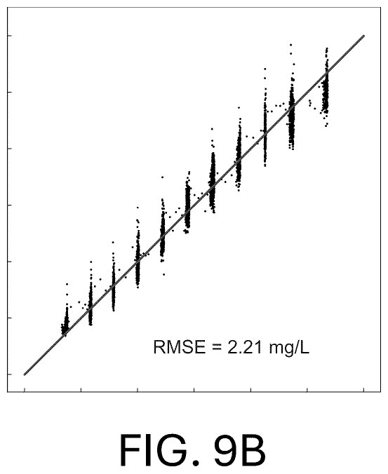

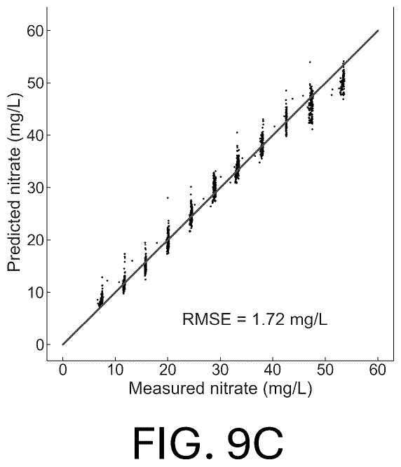

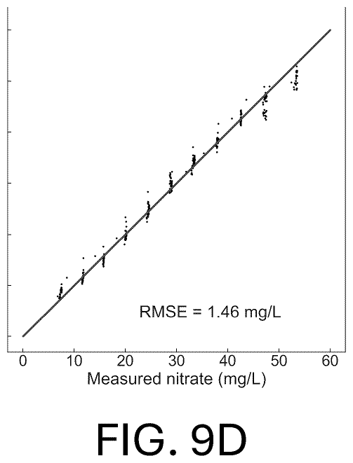

We then examined the improvement gained through averaging the data, seen in FIG. 9 for four different levels of averaging, all using the calibration parameters developed in FIG. 8B. As we noted with the clean water calibration, the improvement due to averaging suggests that improving our sensor's reading protocol would likely improve the device's performance without requiring other modifications.

Finally, we examined the correlation between prediction error and the various parameters of the dirty water test. The only factors with correlations (Pearson's r) with absolute value greater than 0.2 were the right-angle photodiode responses ([r]=0.52 and 0.56 for the 275 nm and 235 nm paths respectively) and nitrate level ([r]=0.20); as a reminder, the right-angle response is the scattered light seen by the other photodiode without direct LED illumination. As we expect the right-angle photodiode response to be related to turbidity, this result suggests that a more thorough examination of turbidity and other matrix effects on our sensor response may improve performance, as expected.

Temperature calibration: Applicants first developed a correction algorithm for the sensor's temperature sensitivity using distilled water as the sample. We started the chamber at ambient temperature, which was around 9° C. in our unheated location. We then set the temperature to 30° C. with the chamber lid ajar to provide a slow temperature ramp. The system recorded a reading every minute for the approximately five hours required to reach the target temperature. We used the recorded data to determine temperature correction curves for each optical path.

Pre-experiment clean water calibration: After the temperature calibration, we derived a calibration curve for the sensor response in distilled water with added sodium nitrate. We performed this calibration five times with the chamber set for different temperatures: 10, 15, 20, 25, and 30° C. At each temperature, we began with 1 L of distilled water and every 40 minutes added a pre-determined quantity of sodium nitrate solution using a computer-controlled dosing pump. The sample container was stirred continuously to ensure thorough mixing. We sampled 16 concentrations from 0 to 111 mg/L of nitrate with particular focus given to the 0-10 mg/L range, which we expected to be the most difficult to sense. Our sensor drew and read a sample every two minutes through the process.

Sensor repair and variance between units: While setting up the next phase of the experiment, the 275 nm photodiode PCB was physically damaged due to an accident. We replaced the board including the photodiode. We used this rebuilt unit for the dirty water experiment. Although inadvertent, this accident gave us better insight into the sensitivity of the sensor to construction and variation between components, which allows us to anticipate the unit-to-unit calibration needs of the design and to plan potential future improvements to reduce this variability.

Dirty water experiment: Next, to evaluate the sensor in a closer setting to real-world conditions, we obtained a five-gallon sample of water from the Mississippi river in urban Minneapolis, USA. We placed a commercial nitrate sensor in the sample to use as a reference (ccoN, OTT HydroMet Corp., Sterling VA, USA). Our sensor drew samples, returning the water to the volume after taking a reading. We protected the sensor's inlet with a section of a mesh filter bag to prevent drawing in large particles.

We added sodium nitrate solution approximately every three days to the sample, increasing the concentration by approximately 5 mg/L per addition as recorded by the reference sensor. We switched the chamber between no heating and 25° C. on a several-day cycle, allowing a gradual heating and cooling to test our sensor's temperature response. Our sensor drew and read a sample every five minutes. We ran the experiment from January 31st to March 5th of 2025.

Post-experiment sensor recalibration: After completing our dirty water experiment, we found that the sensor response appeared to have shifted from our previous calibration. Our sensor repair described above both introduced a replaced photodiode and likely resulted in a different optomechanical alignment, which we believe caused this effect. We performed an abridged recalibration to determine the magnitude of this effect. First, we recorded the sensor response with distilled water across a temperature range from approximately 8° C. to 22° C. Second, we recorded the sensor performance in distilled water with added sodium nitrate solution across seven calculated concentrations from 0 mg/L to 56.6 mg/L, all at a fixed chamber temperature of approximately 15° C. For clarity, we will refer to this data as the post-experiment calibration, and the first calibration as the pre-experiment calibration.

It is possible that some of the response shift resulted from sensor aging. However, as we will discuss, sensor aging or degradation is less likely of a cause than changes resulting from the sensor maintenance. Fully separating these effects will be a priority for future work. We compared the results of the initial calibration and the recalibration to comment on the unit-to-unit variability of the design, and on the calibration needs around sensor maintenance.

All combinations of method or process steps as used herein can be performed in any order, unless otherwise specified or clearly implied to the contrary by the context in which the referenced combination is made. All references to methods or processes are intended to comprise and encompass each embodiment of the compositions described herein, including those described in the examples.

All ranges and parameters, including but not limited to percentages, parts, and ratios, disclosed herein are understood to encompass any and all sub-ranges assumed and subsumed therein, and every number between the endpoints. For example, a stated range of “1 to 10” should be considered to include any and all subranges between (and inclusive of) the minimum value of 1 and the maximum value of 10; that is, all subranges beginning with a minimum value of 1 or more (e.g., 1 to 6.1), and ending with a maximum value of 10 or less (e.g., 2.3 to 9.4, 3 to 8, 4 to 7), and finally to each number 1, 2, 3, 4, 5, 6, 7, 8, 9, and 10 contained within the range.

The systems and corresponding methods of the present disclosure can comprise, consist of, or consist essentially of the essential elements and limitations of the disclosure as described herein, as well as any additional or optional ingredients, components, or limitations described herein or otherwise useful in the general inventive concepts.

The compositions of the present disclosure may also be substantially free of any optional or selected component or feature described herein, provided that the remaining composition still contains all of the required elements or features as described herein. In this context, and unless otherwise specified, the term “substantially free” means that the selected composition contains less than a functional amount of the optional component, typically less than 0.1% by weight, and also including zero percent by weight of such optional or selected component.

To the extent that the terms “include,” “includes,” or “including” are used in the specification or the claims, they are intended to be inclusive in a manner similar to the term “comprising” as that term is interpreted when employed as a transitional word in a claim. Furthermore, to the extent that the term “or” is employed (e.g., A or B), it is intended to mean “A or B or both A and B.” When the Applicant intends to indicate “only A or B but not both,” then the term “only A or B but not both” will be employed. Thus, use of the term “or” herein is the inclusive, and not the exclusive use. In the present disclosure, the words “a” or “an” are to be taken to include both the singular and the plural. Conversely, any reference to plural items shall, where appropriate, include the singular.

In some aspects, it may be possible to utilize the various inventive concepts in combination with one another. Additionally, any particular element recited as relating to a particularly disclosed embodiment should be interpreted as available for use with all disclosed embodiments, unless incorporation of the particular element would be contradictory to the express terms of the embodiment. Additional advantages and modifications will be readily apparent to those skilled in the art. Therefore, the disclosure, in its broader aspects, is not limited to the specific details presented therein, the representative composition, or the illustrative examples shown and described. Accordingly, departures may be made from such details without departing from the spirit or scope of the general inventive concepts.

While the invention has been illustrated and described in detail in the drawings and foregoing description, the same is to be considered as illustrative and not restrictive in character. It should be understood that only the exemplary embodiments have been shown and described and that all changes and modifications that come within the spirit of the invention are desired to be protected.

1. A field-deployable device for analyzing one or more analytes in an aqueous source, the device comprising:
a unit that sits on dry land, comprising a sample chamber and a pump configured to pull sample water into the sample chamber;
a first LED disposed opposite a first photodiode, such that a first path is formed therebetween;
a first focusing lens disposed between the first LED and the first photodiode;
a second LED disposed opposite a second photodiode, forming a second path and oriented such that the second path is perpendicular to the first path;
a second focusing lens is disposed between the second LED and second photodiode;
the sample chamber is disposed at an intersection of the first path and second path, such that light emitted by the first LED and light emitted by the second LED pass through the focusing lenses and the sample chamber before continuing to the first photodiode and second photodiode;
both LEDs and both photodiodes are each in communication with a circuit board;
the first LED is configured to emit light at a predetermined wavelength sensitive to a specific analyte
the second LED is configured to emit light at a predetermined baseline wavelength for the absorbance of the water not including a specific analyte (turbidity and non-target analytes).

2. The field-deployable device according to claim 1, wherein the analyte is nitrate.

3. The field-deployable device according to claim 1, wherein the sample water is selected from surface water and ground water.

4. The field-deployable device according to claim 1, wherein the first LED is configured to emit light at a wavelength of about 220 nm to about 235 nm.

5. The field-deployable device according to claim 1, wherein the second LED is configured to emit light at a wavelength of about 250 nm to about 275 nm.

6. The field-deployable device according to claim 1, further comprising a temperature sensor.

7. The field-deployable device according to claim 1, further comprising a relative humidity sensor.

8. The field-deployable device according to claim 1, wherein the sample chamber is cylindrical.

9. The field-deployable device according to claim 1, further comprising a battery.

10. The field deployable device according to claim 1, further comprising a solar charger.

11. The field deployable device according to claim 1, further comprising a weatherproof enclosure.

12. The field deployable device according to claim 1, further comprising a third LED configured to emit light at a wavelength of about 350 nm.

13. A field deployable system comprising at least two spatially separated devices according to claim 1.

14. A method for analyzing a confined volume of water within a prescribed region of a sample chamber with an optical nitrate sensor, comprising:
pumping a ground water sample from a water source;
confining the ground water sample in a sample chamber positioned within a unit that sits on dry land, wherein the sample chamber sits at an intersection between a first light path from a first light emitter and a second light path from a second light emitter, wherein the second light path is oriented perpendicular to the first light path;
generating a first LED signal at about 220 nm to about 235 nm with a first LED, and a second LED signal in a range of about 250 nm to about 275 nm with a second LED, wherein the first LED signal and the second LED signal traverse the confined volume of water within the prescribed region of the sample chamber;
sensing with a measuring photodiode the first LED signal and the second LED signal;
providing photodiode measured signal containing information about a nitrate absorption of the water related to the first LED signal and the second LED signal;
receiving with a signal processing module the photodiode measured signal containing information from the first LED and the second LED;
calculating the concentration of nitrates dissolved in the water by compensating the concentration of the absorption at about 220 nm to about 235 nm for the absorption at about 250 nm to about 275 nm in order to determine a level of nitrates in the water.

15. The method of claim 14, wherein the measurement is determined from 10 to 100 times per pumping.

16. The method of claim 14, wherein the measurement is determined from 100 times to 1000 times per pumping.

17. The method of claim 14, wherein the calculating determines level of nitrate according to equation 5.

18. The method of claim 14, wherein the calculating comprises determining a level of nitrate at more than one location.

19. The method of claim 14, further comprising compensating for dissolved organic content.
